SEQUENCE LISTING (8 sequences)

SEQ ID NO: 1 - DNA, 564 bp, Mus musculus
atggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaa

SEQ ID NO: 2 - DNA, 564 bp, Cricetulus griseus
atggttcgaccgctgaactgcatcgtcgccgtgtcccagaatatgggcatcggcaagaacggagaccttccctggccaatgctcaggaacgaattcaagtacttccaaagaatgaccaccacctcctcagtggaaggtaaacagaacttggtgattatgggccggaaaacctggttctccattcctgagaagaatcgacctttaaaggacagaattaatatagttctcagtagagagctcaaggaaccaccacaaggagctcattttcttgccaaaagtctggacaatgccttaaaacttattgaacaaccagagttagcagataaagtcgacatggtttggatagttggaggcagttccgtttacaaggaagccatgaatcagccaggccatctcagactctttgtgacaaggatcatgcaggaatttgaaactgacacgttcttcccagaaattgatttggagaaatataaacttctcccagagtacccaagggtccttcctgaagtccaagaggaaaaaggcatcaagtataaatttgaagtctatgagaagaaaggctaa

SEQ ID NO: 3 - DNA, 19 bp, Mus musculus <s300> Wu, Suh-Chin and Hong, Willy W.L. A Novel RNA Silencing Vector to Improve Antigene Expression and Vaccine 25 20 4103-4111 2007-05-16
ggatcatgcaggaatttga

SEQ ID NO: 4 - DNA, 19 bp, Mus musculus <s300> Wu, Suh-Chin and Hong, Willy W.L. A Novel RNA Silencing Vector to Improve Antigene Expression and Vaccine 25 20 4013-4111 2007-05-16
gtacttccaaagaatgacc

SEQ ID NO: 5 - DNA, 19 bp, Mus musculus <s300> Wu, Suh-Chin and Hong, Willy W.L. A Novel RNA Silencing Vector to Improve Antigene Expression and Vaccine 25 20 4103-4111 2007-05-16
ggacagaattaatatagtt

SEQ ID NO: 6 - DNA, 47 bp, Artificial sequence
  chemically synthesized
ggatcatgcaggaatttgacttaagctcaaattcctgcatgatcctt

SEQ ID NO: 7 - DNA, 47 bp, Artificial sequence
  chemically synthesized
gtacttccaaagaatgacccttaagcggtcattctttggaagtactt

SEQ ID NO: 8 - DNA, 47 bp, Artificial sequence
  chemically synthesized
ggacagaattaatatagttcttaagcaactatattaattctgtcctt

(19) United States
(12) Patent Application Publication
(10) Pub. No.: US 2009/0148935 A1
(43) Pub. Date: Jun. 11, 2009

(54) Application of RNA Interference Targeting DHFR Gene, to Cell for Protein Production

(75) Inventors: Suh-Chin Wu, Hsinchu (TW); Willy W.L. Hong, Longtan Township (TW)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: NATIONAL TSING HUA UNIVERSITY, Hsinchu (TW)

(21) Appl. No.: 11/758,203

(22) Filed: Jun. 5, 2007

(51) Int. Cl. C12N 15/64

(52) U.S. Cl. 435/320.1

(57) ABSTRACT

Biological materials are applied to a CHO cell or the like for producing a species of protein. The biological materials includes a first vector and a second vector, the first vector including a dhfr gene of a species of mammal and a gene of the species of protein, the second vector including a DNA fragment for inducing a RNA interference in the CHO cell to reduce expression of a dhfr gene of relevance in said CHO cell after the second vector is applied to the CHO cell. The dhfr gene of the species of mammal, or a dhfr gene of the CHO cell, or both constitute at least part of the dhfr gene of relevance after the first vector is applied to said CHO cell. The DNA fragment consists of nucleotides characterizing a segment of a dhfr gene of the CHO cell and a segment of a dhfr gene of the species of mammal.

### FIELD OF THE INVENTION

The present invention generally relates to application of RNAi (ribonucleic acid interference) to a CHO cell or the like, for producing (expressing) protein.

### BACKGROUND OF THE INVENTION

Mammalian cells have been extensively utilized to produce recombinant proteins as biopharmaceuticals for clinical applications. Amplifiable selective marker such as dihydrofolate reductase (dhfr), and cells such as those from Chinese hamster ovary (CHO), are routinely used to generate stable producer cell clones. Methotrexate (MTX), a folic acid analog which binds and inhibits DHFR, has been widely used to improve recombinant DNA expression in CHO cells by co-amplifying concurrently a target gene and the dhfr gene therein. Stepwise increasing the concentration of MTX in growth medium can result in hundred to thousand copies of the co-amplified target genes in stable producer CHO cells. To date, several attempts have been made to improve the in vitro selection of stable producer CHO cells through stepwise MTX selection, including an internal ribosome entry site (IRES)-driven dicistronic vector, incomplete splicing (in dhfr and target cDNA) vectors, and the use of less-sensitive mutant dhfr genes to MTX However, in vitro selection of high producer cell clones still remains as the most time-consuming process in CHO cell expression technology, and the conventional technologies to produce recombinant proteins is subject to limited efficiency/stability. The stability problem is particularly significant after ending the application of MTX or in MTX-free medium.

RNA interference (RNAi), initially found in Caenorhabditis elegans, has been considered a natural response to double-stranded RNA for controlling sequence-specific gene expression at a post-transcriptional level. Introducing double-stranded RNA in mammalian cells has emerged as a powerful means to silence gene expression in mammalian cells through RNAi. The double-stranded RNAs, transcribed as short hairpin RNA (shRNA) and processed to be active with a length of 19-23 nucleotides by Dicer, can recognize target mRNAs in a sequence-specific manner. Relevant conventional technologies leave a significant margin for improvement.

For more information about relevant RNAi application, reference to inventors' abstract entitled “ENHANCING EXPRESSION OF PROTEIN IN CHO CELLS BY THE CO-AMPLIFICATION OF SPECIFIC shRNA TO DHFR” and posted Jun. 18˜23, 2006 in 20^{th} IUBMB International Congress of Biochemistry and Molecular Biology and 11^{th} FAOBMB Congress, as well as inventors' paper entitled “A novel RNA silencing vector to improve antigene expression and stability in Chinese hamster ovary cells” received Nov. 2, 2006 and to be published in Vaccine Volume 25, Issue 20, May 16, 2007, Pages 4103-4111 by ELSEVIER, shall be made.

### SUMMARY OF THE INVENTION

One of the objects of the present invention is to provide biological materials for applying to at least a cell for promoting production of protein.

Another one of the objects of the present invention is to provide biological materials for applying to at least a target cell for producing protein, with the target cell not necessarily limited to a mutated cell, i.e., either a wild cell or cell line can be used as the target cell.

A further one of the objects of the present invention is to provide biological materials for applying to at least a cell for upgrading the stability of protein production.

One of the advantages of the present invention is that higher production of protein can be achieved.

Another one of the advantages of the present invention is that the cell used for producing protein is not necessarily a mutated cell.

Further one of the advantages of the present invention is that higher stability of protein production can be achieved

Another further one of the advantages of the present invention is that high producer clones can be selected while the probability of successful selection is not lowered.

The present invention features application of biological materials to at least a target cell such as a mammalian cell, for producing a species of protein (i.e., expressing a species of protein, or amplifying gene copies of a species of protein). The biological materials comprise: a first vector including a first gene such as a dhfr gene of a species of mammal, and a second gene of the species of protein (i.e., a second gene encoding the species of protein); and a second vector including a DNA (deoxyribonucleic acid) fragment for inducing, after the second vector is applied to the target cell, a RNA interference in the target cell to reduce (i.e., to silence or inhibit or knock down) expression of a gene of relevance in the target cell.

After applying the first and the second vectors to a target cell, a composition forms to comprise: the first gene, the second gene, and at least a double stranded RNA transcripted in the target cell by the DNA fragment, in addition to the target cell. The composition may be seen as a target cell to which the first and the second vectors have been applied, i.e., a target cell containing the first gene, the second gene, and at least a double stranded RNA transcripted therein by the DNA fragment included in the second vector.

The first gene included in the first vector according to the present invention is usually in the same species as the gene of relevance. For example, if the gene of relevance with expression to be reduced in the target cell is a dhfr (dihydrofolate reductase) gene, the first gene included in the first vector is usually a dhfr gene. The gene of relevance is usually able to resist, at least partially, the chemical material exogenous to the target cell. The selection of the target cell is not limited to cell line, i.e., either cell line or a mutated cell or a wild cell can be selected as the target cell. Specific example of the protein to be produced is an antibody, and the species of the protein to be produced is not limited to antibody. The gene of relevance comprises (or is constituted at least partially by), either the first gene included in the first vector or the gene which is endogenous to the target cell and is in the same species as the first gene included in the first vector. The gene of relevance may also comprise (or be constituted at least partially by) both the first gene included in the first vector and the gene which is endogenous to the target cell and is in the same species as the first gene included in the first vector.

For an aspect of the present invention in which the first vector comprises a gene of a species of mammal and a gene of the species of protein, the selection of the DNA fragment in the second vector, for example, is such that the DNA fragment consists of nucleotides characterizing a segment of a gene of the target cell and a segment of the gene of the species of mammal. Preferably the gene of the species of mammal is in the same species as the gene of the target cell; for example, the nucleotides in the DNA fragment characterize a segment of a dhfr gene of the target cell and a segment of a dhfr gene of the species of mammal. Specifically, the DNA fragment consists of nucleotides which are respectively equivalent to at least part of the nucleotides in the segment of the gene of the target cell, and are respectively equivalent to at least part of the nucleotides in the segment of the gene of the species of mammal included in the first vector. For example, the DNA fragment is designed to consist of a plurality of nucleotides which are respectively equivalent to at least part of the nucleotides in a segment of a dhfr gene of the target cell, and are respectively equivalent to at least part of the nucleotides in a segment of a dhfr gene (in the same species as the dhfr gene of the target cell) included in the first vector. Preferably, if an arbitrary nucleotide in the DNA fragment is equivalent to a nucleotide which is in the segment of the gene of the cell, the arbitrary nucleotide in the DNA fragment is also equivalent to a nucleotide which is in the segment of the gene of the species of mammal and has the same nucleotide sequence as the arbitrary nucleotide. Preferably, the segment or the DNA fragment has a length of at least 19 nucleotides.

For the aspect of the present invention in which the first vector comprises a gene of a species of mammal and a gene of the species of protein, the gene of the species of mammal included in the first vector can be from a mammal which is in the same species as the target cell or in a species different from the species of the target cell.

For a specific aspect of the present invention in which the target cell is a CHO (Chinese hamster ovary) cell and the first vector comprises a dhfr gene of mammal (such as gi:68299777 of mouse) and a gene of the species of protein, the selection of the DNA fragment in the second vector, is such that the DNA fragment consists of nucleotides equal to those which are in a segment of a dhfr gene (such as gi: 191045 or SEQ ID NO: 2) of a CHO cell and are in a segment of the dhfr gene (such as SEQ ID NO: 1) of a mouse. The segment of the dhfr gene of a CHO cell and the segment of the dhfr gene of a mouse, preferably both consist of nucleotides with sequence numbers ranging from 99 to 117 (marked with sd2 as shown in FIG. 1A, i.e., SEQ ID NO: 4), or from 187 to 205 (marked with sd3 as shown in FIG. 1A, i.e., SEQ ID NO: 5), or from 413 to 431 (marked with sd1 as shown in FIG. 1A, i.e., SEQ ID NO: 3), resulting in the fact that an arbitrary nucleotide in the segment of the dhfr gene of a mouse is equal to a nucleotide which is in the segment of the dhfr gene of a CHO cell and has the same sequence number (in the dhfr gene of the CHO cell) as the arbitrary nucleotide has (in the dhfr gene of the mouse). For example, as shown in FIG. 1A, all the nucleotides of segment sd2 (SEQ ID NO: 4) are common to both the dhfr gene of a mouse (SEQ ID NO: 1) and the dhfr gene of a CHO cell (SEQ ID NO: 2), with sequence numbers ranging from 99 to 117 in both the dhfr gene of a mouse and the dhfr gene of a CHO cell.

In this disclosure, “expression vector” corresponds to “first vector” aforementioned, and “silencing vector” corresponds to “second vector” aforementioned.

### Contrast of the Present Invention to Conventional Technologies

An aspect of contrast of the present invention to those representing conventional technologies and characterizing no-use of silencing vector (corresponding to the second vector in the context of the disclosure of the present invention), is typically shown by FIG. 6 (with CHO/dhfr⁻ cells, i.e., dhfr gene deficient CHO cells, as target cells) and FIG. 8 (with dhfr⁻ competent CHO cells as target cells). In FIGS. 6 and 8, E:S=1:0 means the amount ratio of applied expression vector to applied silencing vector is 1:0, E:S=1:1 means the amount ratio of applied expression vector to applied silencing vector is 1:1, and E:S=1:5 means the amount ratio of applied expression vector to applied silencing vector is 1:5, where the expression vector corresponds to the first vector in the context of the disclosure of the present invention, and the silencing vector corresponds to the second vector of the disclosure. The first two digits of the numeric symbols 10-2, . . . , 11-16, . . . , 15-15, etc in FIG. 6 represent the amount ratio of applied expression vector to applied silencing vector, and the number following the first two digits represent the ID of a tested cell clone (or surviving clone). Similarly, the first two digits of the numeric symbols 10-1, . . . , 1-6, . . . ,15-16, etc in FIG. 8 represent the amount ratio of applied expression vector to applied silencing vector, and the number following the first two digits represent the ID of a tested cell (or surviving clone). In FIGS. 6 and 8, the upper diagrams are for illustrating the result of tests where MTX=0, the middle diagrams are for illustrating the result of tests where MTX=1 μM, and the lower diagrams are for illustrating the result of tests where MTX=5 μM; the vertical axis of each diagram means the relative expression of reporter gene(egfp); the horizontal axis of each diagram is marked by numeric symbols 10-2, . . . , 11-6, . . . , 15-17 or 10-1, . . . , 11-6, . . . , 15-16 aforementioned. It can be seen from FIGS. 6 and 8 the protein expression resulting from the application of silencing vector according to the present invention is significantly higher (more than twice) in contrast to those with no-use of silencing vector, regardless of which type of cells are adopted as target cells.

With resultant protein expression significantly higher than conventional technologies, the application of silencing vector according to the present invention, however, does not affect cell survival rate, and may even slightly or significantly increase cell survival rate, as can be seen from FIG. 5 (with CHO/dhfr⁻ cells, i.e., dhfr gene deficient CHO cells, as target cells) and FIG. 7 (with dhfr-competent CHO cells as target cells). In FIGS. 5 and 7, each vertical axis represents cell survival rate(%), each horizontal axis represents concentration (AM) of MTX, the line with marks ♦ or ⋄ represents the case with E:S=1:0, the line with marks ▪ or □ represents the case with E:S=1:1, and the line with marks ▴ or Δ represents the case with E:S=1:5.

Another aspect of contrast of the present invention to those representing conventional technologies and characterizing no-use of silencing vector (corresponding to the second vector in the context of the disclosure of the present invention), is typically shown by FIG. 9. In FIG. 9, the left part and the right part are respectively for illustrating the cases with stable producer cell clones (respectively obtained from CHO/dhFr⁻ cells and CHO-K1 cells) grown in MTX-free medium for two weeks, where the effect of silencing vectors on the stability of EGFP expression represents the effect of silencing vectors on the stability of protein production. In FIG. 9, the left vertical axis means the relative expression of reporter gene, and the right vertical axis means the percentage to the original expression amount (MTX=5 μM) for each clone; E:S=1:0 means the amount ratio of applied expression vector to applied silencing vector is 1:0, E:S=1:1 means the amount ratio of applied expression vector to applied silencing vector is 1:1, and E:S=1:5 means the amount ratio of applied expression vector to applied silencing vector is 1:5; the first two digits of the numeric symbols 10-2, . . . , 11-16 . . . , 15-16, etc represent the amount ratio of applied expression vector to applied silencing vector, and the number following the first two digits represent the ID of a tested cell (or surviving clone).

It can be seen from FIG. 9 that the stability of protein production with application of silencing vector (corresponding to the second vector in the context of the disclosure) according to the present invention can be significantly higher (almost twice) than conventional technologies characterizing no-use of silencing vector.

More about the FIGS. 5-9 will be seen from relevant description below.

Other advantages, objects, and features of the present invention may be seen from the following detailed description with reference to the drawings.

The present invention may best be understood through the following description with reference to the accompanying drawings, in which:

### BRIEF DESCRIPTION OF THE DRAWINGS

FIG. 1A shows nucleotide sequence of dhfr gene of mouse (SEQ ID NO: 1), and nucleotide sequence of dhfr gene of Chinese hamster (SEQ ID NO: 2), wherein “−” represents the nucleotide the same as the one above, i.e., the same as the one with the same sequence number in the sequence of dhfr gene of mouse, and wherein three segments respectively marked with sd1 (SEQ ID NO: 3), sd2 (SEQ ID NO: 4), and sd3 (SEQ ID NO: 5), are to be respectively adopted for designing the DNA fragment included in the second vector according to the present invention.

FIG. 1B shows double stranded RNAs respectively represented by SEQ ID NO: 6, SEQ ID NO: 7, and SEQ ID NO: 8, and respectively corresponding to the gene segments sd1, sd2, and sd3 of FIG. 1A, i.e., the one marked with SEQ ID NO: 6 is designed according to sd1, the one marked with SEQ ID NO: 7 is designed according to sd2, and the one marked with SEQ ID NO: 8 is designed according to sd3.

FIG. 2 is a diagram for illustrating reporting and expression vectors (expression vector corresponds to the first vector according to the present invention, as aforementioned).

FIG. 3 is for illustrating the selection of a silencing vector (silencing vector corresponds to the second vector according to the present invention, as aforementioned).

FIGS. 4A and 4B are for illustrating different amount of DHFR protein and dhfr transcripts, each corresponding to one of different amount levels of silencing vector applied to a target cell according to the present invention.

FIG. 5 is for illustrating the survival rate of CHO/dhfr⁻ cells after different amount of silencing vectors are applied according to the present invention.

FIG. 6 is for illustrating protein expression in CHO/dhfr⁻ cells for different amount of silencing vectors applied thereto and for the application of different amount of MTX.

FIG. 7 is for illustrating the survival rate of dfhr-competent CHO-K1 cells after different amount of silencing vectors are applied according to the present invention.

FIG. 8 is for illustrating protein expression in dhfr-competent CHO-K1 cells for different amount of silencing vectors applied thereto and for the application of different amount of MTX.

FIG. 9 is for illustrating stability of protein production CHO cells for different amount of silencing vectors applied thereto.

FIG. 10 is for illustrating IgG secretion of stable clones.

FIG. 11 is for illustrating stability of secretary IgG produced in CHO cells.

### DETAILED DESCRIPTION OF THE PREFERRED EMBODIMENTS/PRESENT INVENTION

### Materials and Methods

### Construction of Plasmids (i.e., Vectors)

Expression vectors (pEGFP-DHFR) were generated by replacing the neomycin phosphotransferase gene (neo) gene with mouse dhfr cDNA driven by an SV40 promoter in the pcDNA3.1(+) (Invitrogen). The EGFP fragment from pEGFP-N1 (BD Biosciences), driven by cytomegalovirus (CMV) immediate-early gene promoter and enhancer was cloned into BamHI and EcoRI sites. The internal ribosomal entry site (IRES) on pIRES (BD Biosciences) and the Zeocin (Zeo) gene from pcDNA3.1/Zeo (Invitrogen) were cloned into XhoI/XbaI and XbaI/ApaI sites, respectively, to select colonies. The human polymerase-III U6 promoter was amplified from the genomic DNA of HeLa cells (ATCC, CCL-2) and cloned in front of the mouse/hamster dhfr-specific shRNA with five thymidines as a terminator signal to construct shRNA silencing vectors (pCMVen-sd1, pCMVen-sd2, and pCMVen-sd3). The CMV promoter and the enhancer were replaced with an CMV enhancer upstream of the U6 promoter to increase the efficacy of silencing in the identification of shRNA candidates. The control vector (pCMVen) has the same CMV enhancer/U6 promoter with no shRNA. The antibody vector (IgG-expression vector) for chimeric full-length IgG1 expression was constructed from plasmid pFab CMV-dhfr 2H7, containing a dual expression cassette with CMV promoters and poly(A) site for the heavy chain and light chain of IgG, DHFR expression cassette, and neo and □-lactamase (amp) genes. The DNA fragments of variable regions of heave chain (V_{H}) and light chain (V_{L}) from Japanese encephalitis virus-neutralizing antibody, E3.3, were used to replace those in the pFab CMV-dhfr 2H7 and generate a chimeric whole IgG1 expression vector.

### Cell Culture and Transfection

Wild-type CHO-K1 cells (ATCC, CCL-61) and the dhfr-deficient mutant CHO/dhFr⁻ cells (ATCC, CRL-9096), to be used as target cells, were obtained from FIRDI (Taiwan) and cultured at 37° C. in a humidified incubator with 5% CO₂. CHO-K1 and CHO/dhFr⁻ cells, before transfection and cloning selection, were maintained in Ham's F-12K and MEM-α media supplemented with 10% fetal bovine serum (FBS) (Invitrogen), 100 units/ml penicillin, and 100 μg/ml streptomycin (Invitrogen). CHO cells were transfected with expression and silencing vectors using Lipofectamine 2000 (Invitrogen).

### Western Blotting

CHO cells that had been transfected with reporting and silencing vectors were harvested. Equal amounts of protein were resolved by SDS-PAGE (12% polyacrylamide) and transferred to a nitrocellulose membrane (Millopore) by electroblotting. Immune complexes were visualized with BCIP/NBT substrate kit (Invitrogen) using anti-DHFR IgG (BD Biosciences), anti-GAPDH IgG (Abcam) and Anti-mouse IgG-AP.

### Stable Clone Selection and Gene Amplification

The CMV enhancer was removed before transfection to prevent interference by the CMV enhancer of CMV enhancer-promoter-dependent transcription. The linearized plasmids (2 μg) were co-transfected into CHO-K1 and CHO/dhFr⁻ cells with a 1:0, 1:1 or 1:5 ratio of the expression vector (pEGFP-DHFR) to the psd2 silencing vector that contained only CMV promoter without the enhancer. Following transfection, the cells were transferred into two 60 mm plates, grown for two days in a nonselective growth medium, and then replaced the medium with ribonucleosite/deoxyribonucleosite-free MEM-α (Invitrogen) with 10% dialyzed serum (DS, Invitrogen), 200 μg/ml Zeocin (Invitrogen) and 600 μg/ml G-418 (Calbiochem) to select DHFR-, Neo-, and Zeo-positive colonies. Only Zeocin was used to select colonies of the control transfected cells (pEGFP-DHFR only). Ten to 14 days after the cells were transferred to the selective medium, a limiting dilution in the 96-well plates was employed to isolate cell clones and the wells that contained single cells were labeled under fluorescent microscopy. Single cell clones were isolated and 18 of the colonies that expressed the highest level of EGFP or IgG were employed in the subsequent methotrexate (MTX)-driven amplification. The MTX concentration in CHO cell culture medium was increased from 0.04 μM to 1 μM (IgG-expressed clones) or 5 μM (EGFP expressed clones). In each selection step, cells were cultivated for at least 15 days before the MTX concentration was increased.

### Real-Time PCR and RT-PCR

The genomic DNA extraction from stable CHO cell clones was performed using DNeasy Tissue kit (Qiagen). The RNAs were extracted from the transfected cells using Trizol (Invitrogen). The primers and Taqman probe (Applied Biosystems assay ID 293340) were employed for real-time PCR measurement to determine the copy numbers of mouse dhfr gene. One-step RT-PCR was performed to obtain the amount of RNA transcripts of mouse and hamster dhfr genes. The real-time PCR and RT-PCR assays were performed on an ABI PRISM 7500 real time PCR system, by calculating the absolute amount of DNA and the relative amount of RNA by the ΔΔCt method, while the amounts of RNA transcripts were normalized to that of eukaryotic 18s rRNA.

### Enzyme-Linked Immunosorbent Assay (ELISA)

IgG concentration in culture supernatants was determined according to ELISA. Specific productions were determined at 72 h post cell inoculation. Plates (Coring, 9018) were pre-coated with domain III of JEV envelop proteins and the bound antibodies quantified with a anti-human-Fc antibody-HRP and TMB substrate (Pierce). To calculate the specific productions, the total IgG produced was divided by the integral of the total cell number and 3 (3 days). Results are given as pg/cell/day.

### Results

Silencing Vector Targeting dhfr Gene Expression in CHO/dhFr⁻ Cells

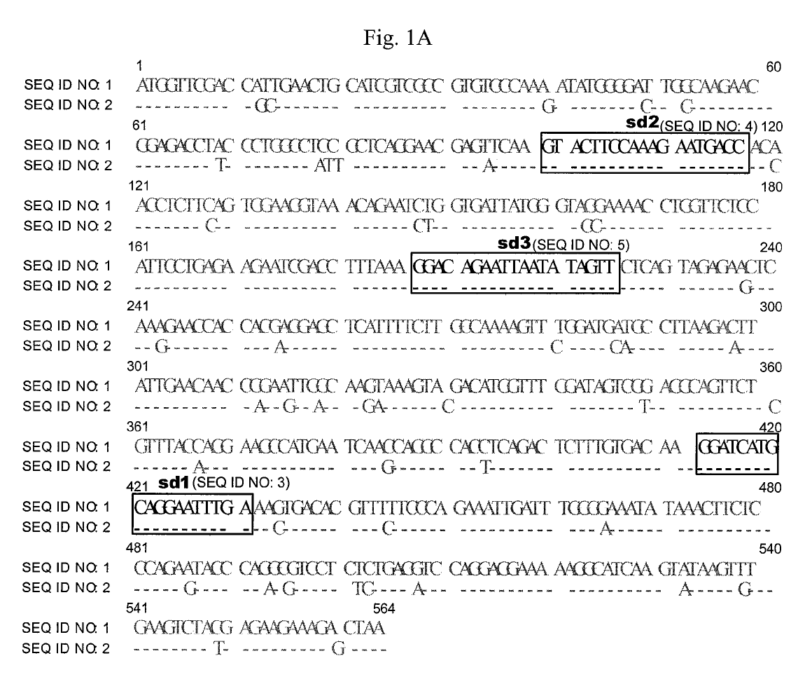

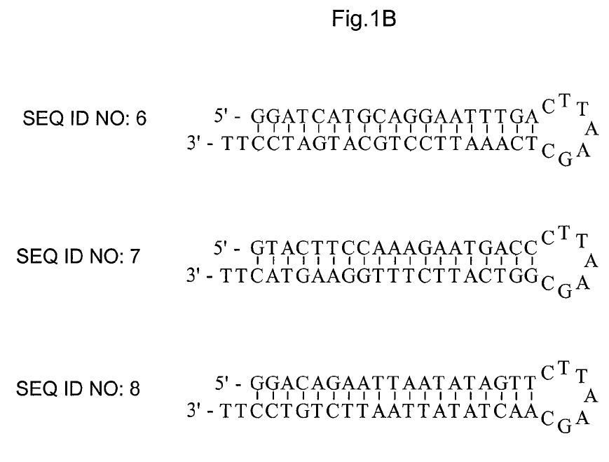

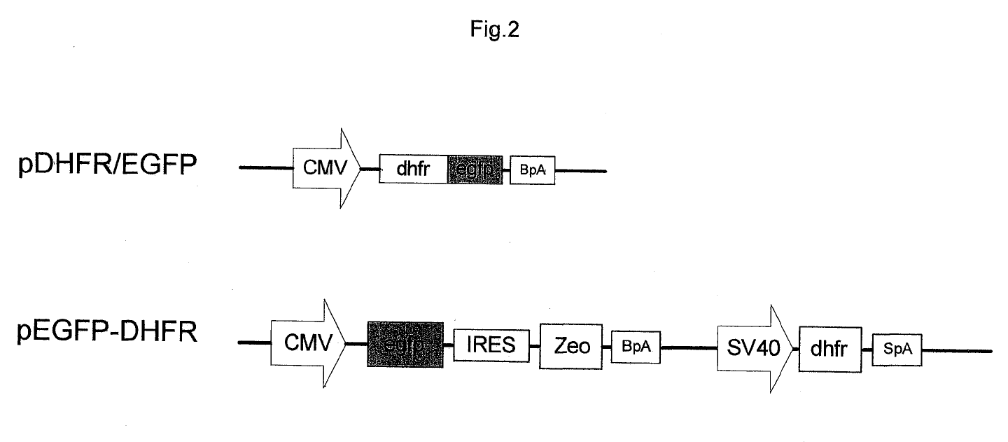

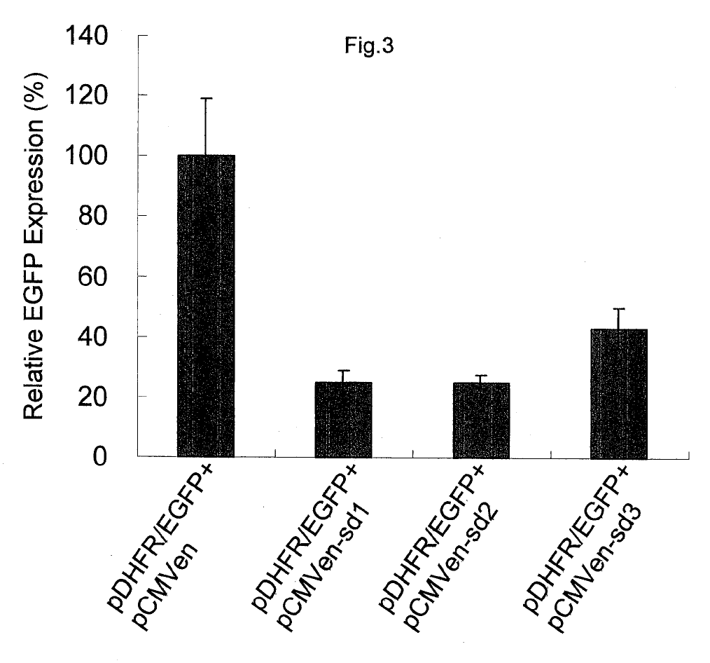

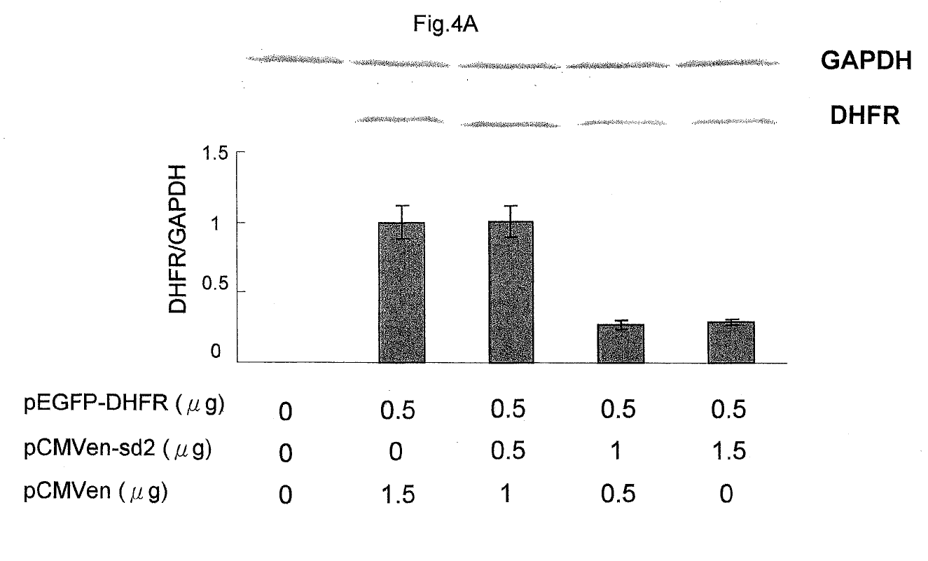

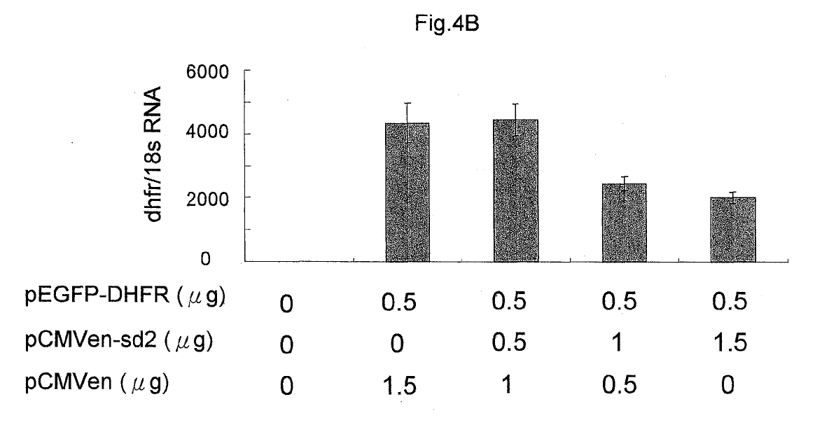

Three sequence segments GT ACTTCCAAAG AATGACC (SEQ ID NO: 4, marked with sd2 in FIG. 1A), GGAC AGAATTAATA TAGTT (SEQ ID NO: 5, marked with sd3 in FIG. 1A), and GGATCATG CAGGAATTTG A (SEQ ID NO: 3, marked with sd1 in FIG. 1A), located in the conserved sequences of the mouse and Chinese hamster dhfr genes, as shown in FIG. 1A, were respectively selected for constructing three different silencing vectors, to silence or reduce or inhibit or knock down exogenous (mouse) and/or endogenous (Chinese hamster) dhfr genes in CHO/dhFr⁻ and CHO-K1 cells. The three silencing vectors, respectively representing a different one of three shRNAs which respectively correspond to the sd1 (SEQ ID NO: 3), sd2 (SEQ ID NO: 4), and sd3 (SEQ ID NO: 5), and driven by human polymerase-III U6 promoter and the CMV enhancer, were constructed (as shown in FIG. 1B). To evaluate the effects of dhfr-silencing, reporting vector (pDHFR/EGFP), encoding the fusion dhfr-egfp gene, and expression vectors (pEGFP-DHFR), encoding the egfp and dhfr genes under two different promoters, were also constructed (FIG. 2). The silencing vectors, pCMVen-sd1, pCMVen-sd2 and pCMVen-sd3, were co-transfected with the reporting vector pDHFR/EGFP in CHO/dhFr⁻ cells, resulting in the reduction of EGFP expression levels by 74% (pCMVen-sd1), 75% (pCMVen-sd2) and 57% (pCMVen-sd3) (as shown in FIG. 3) as determined by using Wallac Victor2 Multicounter (PerkinElmer Life Science). Dose dependent experiments showed that 1 μg and 1.5 μg of the silencing vector pCMVen-sd2 resulted in a maximum of reducing EGFP expression by over 70% (normalized to GAPDH) (as shown in FIG. 4A), correlated with the reduction of dhfr RNA transcripts by around 60% (normalized to 18 sRNA), as shown in FIG. 4B. The silencing vector pCMVen-sd2 was demonstrated to be most effective to silence dhfr RNA transcripts and knockdown EGFP gene expression under the conditions tested.

Silencing Vector Targeting dhfr Gene to Improve the Selection of Stable Producer Clones of CHO/dhFr⁻ Cells Through Stepwise MTX Selection

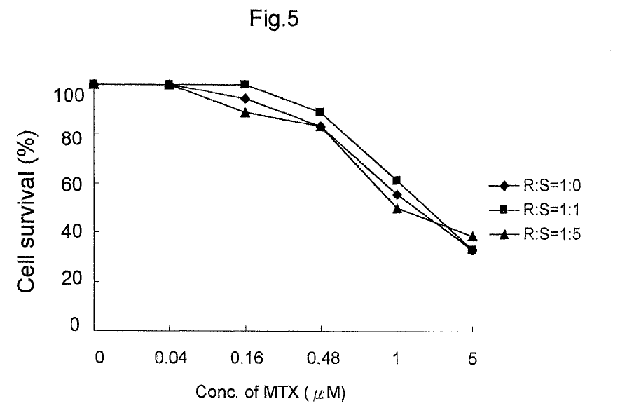

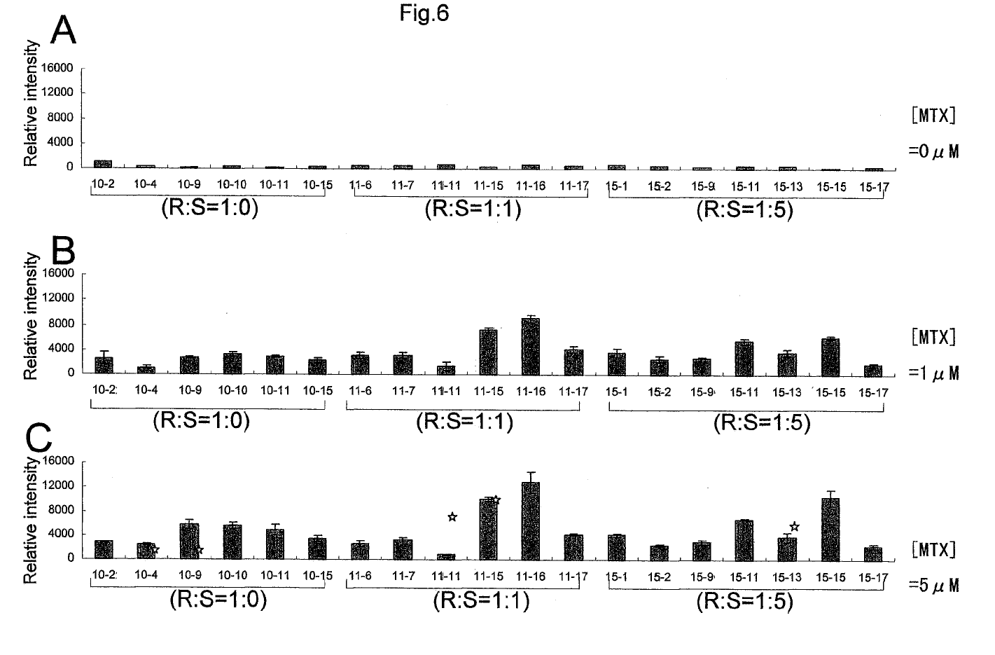

The amplification of the targeted dhfr gene in CHO/dhFr⁻ cells can be obtained by stepwise MTX selection. The expression vector (PEGFP-DHFR) and the silencing vector psd2 (pCMVen-sd2 without CMV enhancer) were mixed in various ratios (E:S=1:0, 1:1, and 1:5); transfected to CHO/dhFr⁻ cells, and then underwent G418 and Zeocin selection, to study the effects of dhfr-targeted RNA silencing vectors on target gene amplification in CHO/dhFr⁻ cells through stepwise MTX selection. Approximately 80 cell clones were obtained from the single-cell cloning of G418/Zeocin-resistant pool cells, and a total of 19 cell clones finally survived the stepwise MTX selection. The use of psd2 silencing vectors did not influence the cell survival rates of CHO/dhFr⁻ cells during stepwise-increased MTX selection (FIG. 5). The stepwise MTX selection process in the CHO cell culture significantly enhanced EGFP expression, as presented in these 19 stable clones at 0 μM, 1 μM and 5 μM MTX (FIG. 6). The use of the sd2 silencing vector resulted in the selection of three high-producer cell clones, 11-15, 11-16 and 15-15, which exhibited an EGFP expression that was over 100% more than those of two high producer clones, 10-9 and 10-10, selected without the use of silencing vector (FIG. 6). Quantitative PCR was further used to determine the numbers of co-amplified gene copies of five high-producing stable clones of CHO/dhFr⁻ cells. Without the use of psd2 silencing vectors, the number of dhfr gene copies were 33.0±4.8 for clone 10-9 and 15.0±2.9 for clone 10-10 at 0 μM MTX, increasing to 233.3±16.3 for clone 10-9 and 458.6±24.8 for clone 10-10 at 5 μM MTX. Stable clones obtained using psd2 silencing vector yielded the number of gene copies equal to 5.1±0.6 for clone 11-15, 2.2±0.1 for clone 11-16, and 1.6±0.3 for clone 15-15 at 0 μM MTX, increasing to 387.1±22.6 for clone 11-15, 442.1±31.5 for clone 11-16 and 240.5±29.2 for clone 15-15 at 5 μM MTX. The results indicated that these high-producing stable clones formed with and without the use of silencing vector for stepwise MTX selection yielded similar numbers of copies ranging from 200 to 400 at 5 μM MTX. Therefore, the use of sd2 silencing vector improves the selecting of stable cell clones with enhanced target gene expression but not the number of co-amplified gene copies by stepwise MTX selection.

Silencing Vector Targeting dhfr Gene to Improve the Selection of Stable Producer Clones of Cho-K1 Cells Through Stepwise MTX Selection

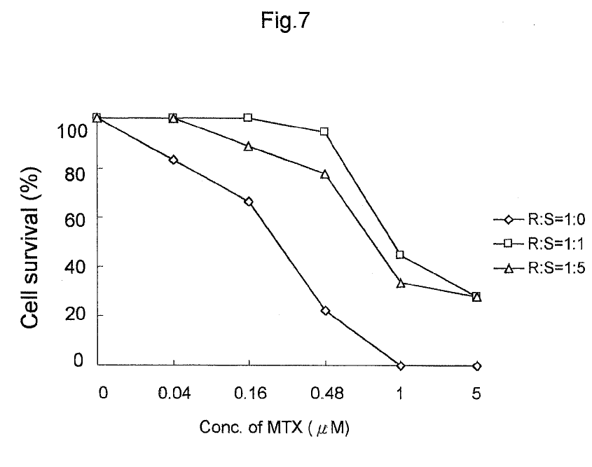

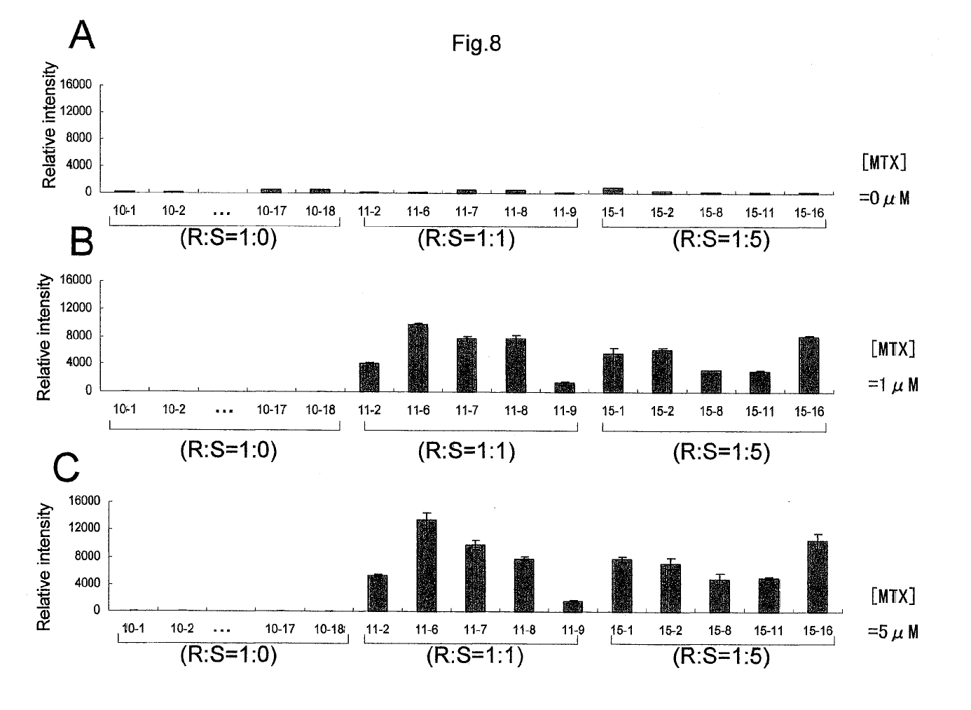

CHO-K1 cells is generally subject to lower frequency of obtaining stable producer cell clones by DHFR/MTX gene amplification since the endogenous dhfr gene in CHO-K1 cells can resist MTX selection. The results reveal that CHO-K1 cells transfected with 1 g and 1.5 μg of the pCMVen-sd2 silencing vector reduced the amount of endogenous dhfr RNA transcripts by 40%-50% and the endogenous DHFR expression by 70%-80% (data not shown). Whether or not the use of sd2 silencing vector increased the dhfr-directed gene amplification in CHO-K1 cells through stepwise MTX selection was investigated. The sd2 silencing vector was mixed with the expression vector pEGFP-DHFR in various ratios (E:S=1:0, 1:1 and 1:5) and co-transfected into CHO-K1 cells, before undergoing stepwise MTX selection as previously performed in CHO/dhFr− cells. As expected, stable producer CHO-K1 cell clones obtained without the use of sd2 silencing vector did not survive the stepwise MTX selection at 0.48 μM (FIG. 7). Around 20% CHO-K1 stable producer cell clones survived at MTX=5 μM (FIG. 7). The CHO-K1 stable clones (11-6, 11-7, 11-8, 15-1 and 15-16) exhibited enhanced EGFP expression as MTX increased to 1 μM and 5 μM (FIG. 8).

### Stability of Stable Producer CHO/dhFr⁻ and CHO-K1 Cells in MTX-Free Medium

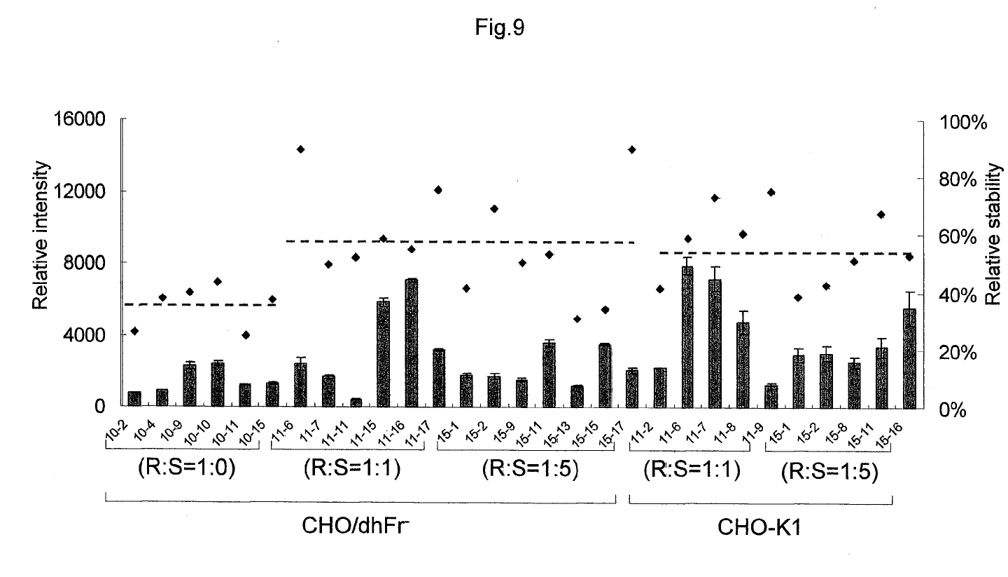

All stable producer cell clones obtained from CHO/dhFr− and CHO-K1 cells were grown in the MTX-free medium for two weeks to elucidate the effect of sd2 silencing vectors on the stability of EGFP expression in stable producer CHO cells. The results indicated that EGFP expression was reduced in CHO/dhFr⁻ and CHO-K1 stable producer cell clones by 30% to 90% in MTX-free medium (FIG. 9). However, with the use of sd2 silencing vectors, on average, the relative stability of EGFP expression in CHO/dhFr− stable clones was 63.32%, and in CHO-K1 stable clones was 56.76%, which values compare to 34.84% in CHO/dhFr⁻ stable clones obtained without the use of sd2 silencing vector (E:S=1:0, FIG. 9). Notably, the stable producer cell clones CHO/dhFr⁻-11-15, CHO/dhFr⁻-11-16, CHO/dhFr⁻-15-17, CHO-K1-11-6, CHO-K1-11-7 and CHO-K1-15-16 had a relative stability of over 53% EGFP expression. These results indicated that the use of sd2 silencing vector also increased the stability of stable producer CHO cells clones for recombinant DNA expression.

Applications of Silencing Vector Targeting dhfr Gene for Secretary IgG Expression in Stable Producer Cho/dhfr⁻ and CHO-K1 Cells

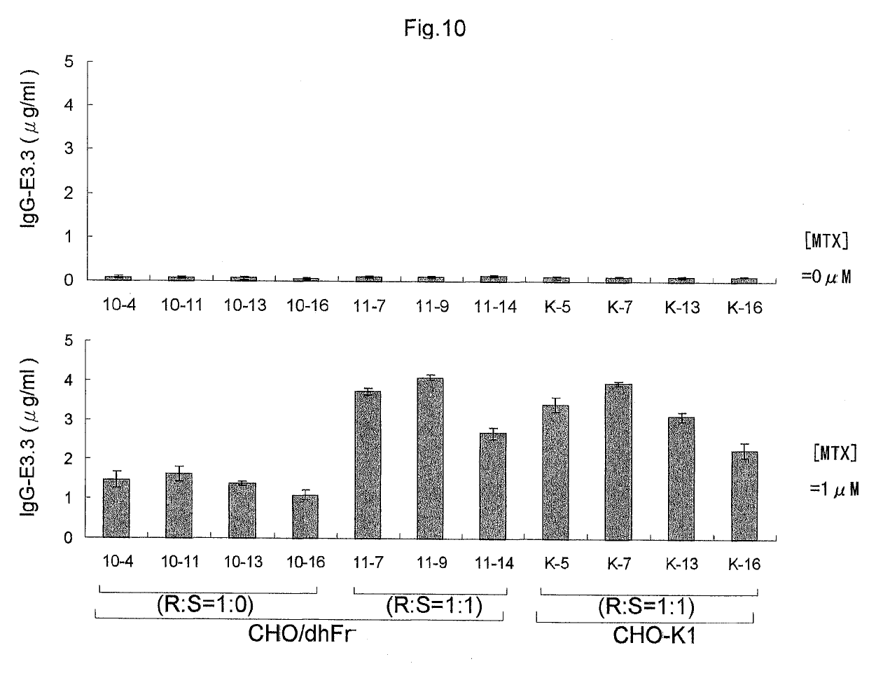

The sd2 silencing vector was further applied for the expression of a chimeric IgG1-E3.3 in CHO/dhFr− and CHO-K1 cells to obtain stable producer cell clones through stepwise MTX selection. The expression vector contained heavy and light chains of a chimeric IgG1-E3.3 targeted on domain III of the Japanese encephalitis virus envelope protein and the dhfr gene under three separate promoters. The sd2 silencing vector was co-transfected with the IgG1-E3.3 expression vector into CHO/dhFr⁻ cells and CHO-K1 cells at E:S ratios of 1:0 and 1:1 (FIG. 10). Approximately 80 single cell clones were chosen from the pool of each transformant and 16 highly expressed clones were obtained throughout stepwise MTX selection (0.04 μM→0.16 μM→0.48 μM→1.0 μM). As shown in FIG. 10, the secretion of IgG-E3.3 in these surviving cell clones increased with the MTX concentration from 0 μM to 1 μM. The use of sd2 silencing vector resulted in the selection of five high producer cell clones, 11-7, 11-9, K-5, K-7 and K-13, which exhibited a secretary IgG expression that is more than 100% higher than that of the producer clones selected without a silencing vector (FIG. 10). The yields obtained in high-producing clones using sd2 silencing vector were 8.9-13.6 pg/cell/day (CHO/dhFr⁻ cells) and 7.5-13.2 pg/cell/day (CHO-K1 cells), compared to 3.6-5.4 pg/cell/day (CHO/dhFr⁻ cells without silencing vector).

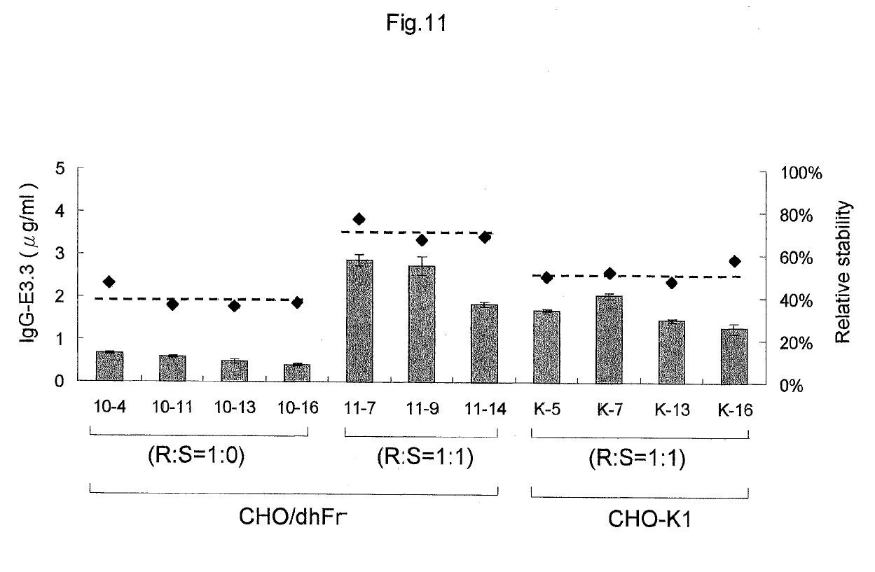

All stable producer cell clones obtained were also grown in the MTX-free medium to elucidate the effect of sd2 silencing vector on the stability of IgG expression in stable producer clones. The results indicated that secretary IgG expression was reduced in stable producer cell clones by 30% to 80% in MTX-free medium (FIG. 11). However, with the use of sd2 silencing vector, on average, the relative stability of IgG expression in CHO/dhFr⁻ stable clones was 70.82%, and in CHO-K1 stable clones was 51.70%, which values compare to 39.03% in CHO/dhFr⁻ stable clones obtained without the use of sd2 silencing vector (FIG. 11). These results further demonstrated that the sd2 silencing vector enhanced the expression of secretary IgG in stable clone through MTX gene amplification and the stability of stable producer CHO cells clones for IgG expression.

### Remarks

Among the advantages of using silencing vectors to form shRNA targeting dhfr gene are powerful inducer, high transfection efficiency, low cytotoxicity, and the dhfr-directed target gene co-amplification throughout stepwise MTX selection. In this study, we developed shRNA silencing vectors respectively constructed on the basis of segments sd1, sd2, sd3 targeting the conserved sequences of the mouse and Chinese hamster dhfr genes. Our results demonstrated that the silencing vectors targeting dhfr gene, not only induced effective shRNA against the dhfr gene transcripts, but also yielded a permanent integration of a silencing cassette, resulting in co-amplification of the dhfr-directed target gene throughout stepwise MTX selection to increase the strength of RNAi specific to the dhfr gene transcripts.

The dhfr-directed target gene amplification requires the stepwise increase of MTX concentration corresponding to the increased DHFR expression for subsequent cell growth. As the amplification of the endogenous dhfr gene inevitably results in a low frequency of transfection and gene amplification, only a few reports documented the amplification of the exogenous dhfr gene in wild-type CHO cells. As demonstrated herein, the lower, but still present, expression of endogenous dhfr gene in CHO-K1 cells suffices for generating a highly producing stable cell line by gene amplification. The knockdown of endogenous dhfr gene expression in CHO-K1 cells in this paper resembled that in the cellular environment of the CHO/dhFr− cells, and this down-regulation is sufficient for exogenous dhfr gene amplification. Therefore, the silencing cassette in psd2 that is specific to both exogenous (mouse) and endogenous (Chinese hamster) dhfr genes was co-transfected into CHO-K1 cells and similar productivity (as determined by comparing FIGS. 6 and 8), similar efficiency of transfection (FIG. 5) and similar production stability (FIG. 9) were achieved. Whether or not the co-amplification in CHO-K1 cells mediated by the silencing vector, can be applied to other dhfr-competent cell lines, remains unknown. This result suggests the potential of using other well-characterized cell lines that contain an unstable karyotype to produce therapeutic recombinant proteins through gene amplification.

The stability of the stable producer cell clones is also important for CHO cell expression of recombinant proteins. Unstable CHO cell karyotypes formed by translocation and homogenous recombination usually resulted in decrease of target gene expression particularly in cell cultures with MTX removal. We observed a 60-65% drop in the EGFP and secretary IgG expression in stable producer clones of CHO/dhFr− cells during the first two weeks without silencing vector (FIGS. 9 and 11). Notably, the dhfr-targeted sd2 silencing vector retains 13-36% higher stability in stable producer clones of CHO/dhFr− and CHO-K1 cells with MTX removal. This difference arises presumably because RNAi can suppress the generation of some redundant DHFR at a high concentration of MTX under culturing conditions without selective pressure, so the number of lost copies of the gene is less than that in the absence of the silencing vector during a two-week culture. The integration and subsequent amplification of the exogenous gene in genome are known to be critical to the long-term stability of the culture. The integrated location of the host chromosome and epigenetic repression also influence the stability. Given the factors described above, the position effect probably explains the instability in subclones for a 60-generation culture (data not shown).

In conclusion, the strategy, applied herein, of combining the dhfr silencing vector with the transgene-expressing vector through stepwise gene amplification, can generate strongly expressed cell clones in CHO/dhFr⁻ and CHO-K1 cells with equally efficient stable transfection. This method was also applied to obtain the higher-producing clone for secretory IgG1 expression. Silencing vectors again increased the IgG1 stability in the absence of MTX. The utilization of the silencing vector may open up new means of investigating gene amplification in other wild-type (dhfr-competent) cell lines for the expression of recombinant proteins.

### DRAWING LEGENDS

FIGS. 1A, 1B

FIG. 1A shows sequence alignment analysis of the dhfr gene and three selected segments respectively for constructing different DNA fragments each for a silencing vector. The number of dhfr genes of mouse (SEQ ID NO: 1) and Chinese hamster (SEQ ID NO: 2) are shown and the consensus nucleotides symbolized as dash. Three segments (respectively represented by sd1 (SEQ ID NO: 3), sd2 (SEQ ID NO: 4), and sd3 (SEQ ID NO: 5)) of 19 consensus nucleotides behind two adenines(A), each selected for designing a shRNA, was respectively indicated in three rectangles. FIG. 1B shows what are included in the double-stranded RNAs supposed to be transcripted in a target cell to which the silence vector is applied or transfected, wherein the double-stranded RNA marked with SEQ ID NO: 6 is designed according to sd1, the one marked with SEQ ID NO: 7 is designed according to sd2, and the one marked with SEQ ID NO: 8 is designed according to sd3.

FIG. 2

Schematic drawing of reporting and expression vectors. The reporting vector (pDHFR/EGFP) containing DHFR/EGFP fusion protein and expression vector (pEGFP-DHFR) containing the egfp and dhfr genes under two separate promoters: CMV and SV40, were shown here. EGFP, enhanced green fluorescent protein; IRES, internal ribosome entry site from encephalomyocarditis virus; Zeo, zeocin resistance gene; BpA, bovine growth hormone polyadenylation signal; SV40, SV40 early promoter; dhfr, dihydrofolate reductase gene of mouse; SpA, SV40 early polyadenylation signal.

FIG. 3

Screening of silencing vectors to dhfr gene. One μg of reporting vector (pDHFR-EGFP) and two μg of each silencing (pCMVen-sd1, pCMVen-sd2, and pCMVen-sd3) or control (pCMVen, without shRNA) vector were co-transfected into CHO/dhFr⁻ cells to silence the DHFR/EGFP fusion protein; the fluorescence was determined using a Wallac Victor2 Multicounter and the relative fluorescence were indicated. The experiments were performed in triplicate; the means (i standard deviation) of EGFP fluorescence intensity are presented.

FIGS. 4A and 4B

RNA silencing to dhfr gene in CHO/dhFr⁻ cells. CHO/dhFr⁻ cells were transfected with increasing amounts of pCMVen-sd2 and the same amount of expression vector (pEGFP-DHFR), which expresses mouse dhfr gene and was used for the following experiments. Fifty hours following transfection, the cells were harvested and separated on 12% SDS-PAGE. Immunoblotting with DHFR-specific antibody was performed and the bands that correspond to the DHFR protein and an internal control (GAPDH) are indicated. The ratio of DHFR expression to GAPDH expression was determined using Gel-Pro 3.1, as shown in FIG. 4A. The amount of dhfr transcript from transfected cells was also measured by quantitative real-time RT-PCR and 18s rRNA is used as an internal control for normalization, as shown in FIG. 4B.

FIG. 5

Survival rate of transfected CHO/dhFr⁻ cells during stepwise MTX selection/amplification. Survival rate of clones from CHO/dhFr⁻ with different ratio of expression vectors and silencing vectors were calculated. The data are shown as the percentage of clones surviving at the indicated concentration of MTX relative to the original clones. The expression vectors (pEGFP-DHFR, E) and silencing vectors (psd2, S) vectors in different ratios (1:0, 1:1, and 1:5) were indicated.

FIG. 6

Co-transfection of silencing vector increases protein expression in CHO/dhFr⁻ cells. The linearized vectors were transfected into CHO/dhFr⁻ cells, and 18 colonies that most strongly expressed EGFP were isolated. During the stepwise increase in the concentration of MTX in the growth medium, the fluorescent intensity of EGFP expressed in survival colonies at MTX concentrations of 0 (A), 1.0 (B) and 5.0 μM (C) were measured. The expression vectors (pEGFP-DHFR, E) and silencing vectors (psd2, S) vectors in different ratios (1:0, 1:1, and 1:5) were introduced into CHO/dhFr⁻ cells as indicated.

FIG. 7

Survival rate of transfected CHO-K1 cells (dhfr-competent cells) during stepwise MTX selection/amplification. Survival rate of clones from CHO-K1 with different ratio of expression vectors and silencing vectors were calculated. The data are shown as the percentage of clones surviving at the indicated concentration of MTX relative to the original clones. The expression vectors (pEGFP-DHFR, E) and silencing vectors (psd2, S) vectors in different ratios (1:0, 1:1, and 1:5) were indicated.

FIG. 8

Co-transfection of silencing vector increases protein expression in dhfr-competent CHO-K1 cells. The linearized vectors were transfected into CHO-K1 cells, and 18 colonies that expressed the highest EGFP levels were isolated. After the MTX concentration was stepwise increased in the growth medium, the fluorescent intensity of EGFP was measured (A, B, and C). The expression vectors (pEGFP-DHFR, E) and silencing vectors (psd2, S) vectors in different ratios (1:0, 1:1, and 1:5) were introduced into CHO/dhFr⁻ cells, as indicated. Transformants that contained pEGFP-DHFR vector only could not endure an MTX pressure of over 1.0 μM, so no CKO-K1 (1:0) clone is presented here.

FIG. 9

Stability of recombinant proteins produced in CHO cells. Fluorescence of EGFP expressed in transformants under culture conditions without selection pressure (MTX). After stepwise amplification in 5 μM MTX, each surviving clone was transferred to the fresh growth medium without MTX, and subcultured five times in 15 days. The figure presents the final fluorescence and the percentage to the original value (MTX=5 μM, black diamond) for each clone.

FIG. 10

The IgG secretion of the stable clones. Three-days after inoculation, the concentration of antibody in the supernatant of clones expressing E3.3 at MTX concentrations of 0 and 1.0 μM were determined by ELISA. The IgG-expression vectors (E) expressing IgG and silencing vectors (psd2, S) vectors in different ratios (1:0, 1:1, and 1:5) were indicated. The specific productions pg/cell/day) were calculated and shown here.

FIG. 11

Stability of secretory IgG produced in CHO cells. Concentration of IgG expressed in transformants under culture conditions without selection pressure (MTX). Each surviving clone in 1 μM MTX was transferred to the fresh growth medium without MTX and subcultured five times in 15 days. The figure presents the final concentration and the percentage to the original value (MTX=1 μM, black diamond) for each clone.

While the invention has been described in terms of what are presently considered to be the most practical and preferred embodiments, it shall be understood that the invention is not limited to the disclosed embodiment. On the contrary, any modifications or similar arrangements shall be deemed covered by the spirit of the present invention.

What is claimed is:

1. A plurality of biological materials for applying to at least a CHO cell for producing a species of protein, comprising:
a first vector including a dhfr gene of a species of mammal, and a gene of said species of protein; and
a second vector including a DNA fragment for inducing a RNA interference in said CHO cell to reduce expression of a dhfr gene of relevance in said CHO cell after said second vector is applied to said CHO cell.

2. The plurality of biological materials according to claim 1 wherein said dhfr gene of said species of mammal is a dhfr gene of a mouse.

3. The plurality of biological materials according to claim 1 wherein said dhfr gene of said species of mammal is a mutated gene.

4. The plurality of biological materials according to claim 1 wherein said gene of said species of protein is a gene of an antibody.

5. The plurality of biological materials according to claim 1 wherein said dhfr gene of said species of mammal constitutes part of said dhfr gene of relevance after said first vector is applied to said CHO cell.

6. The plurality of biological materials according to claim 1 wherein a dhfr gene of said CHO cell is part of said dhfr gene of relevance.

7. The plurality of biological materials according to claim 1 wherein said dhfr gene of said species of mammal and a dhfr gene of said CHO cell constitute at least part of said dhfr gene of relevance after said first vector is applied to said CHO cell.

8. The plurality of biological materials according to claim 1 wherein said DNA fragment consists of nucleotides characterizing a segment of a dhfr gene of said CHO cell and a segment of a dhfr gene of said species of mammal.

9. The plurality of biological materials according to claim 8 wherein an arbitrary nucleotide in said segment of said dhfr gene of said species of mammal is equivalent to a nucleotide which is in said segment of said dhfr gene of said CHO cell and has the same sequence number in said dhfr gene of said CHO cell as said arbitrary nucleotide has in said dhfr gene of said species of mammal.

10. The plurality of biological materials according to claim 8 wherein said segment has a length of at least 19 nucleotides.

11. The plurality of biological materials according to claim 8 wherein said segment consists of nucleotides with sequence numbers ranging from 99 to 117 in said dhfr gene of said species of mammal.

12. The plurality of biological materials according to claim 8 wherein said segment consists of nucleotides with sequence numbers ranging from 187 to 205 in said dhfr gene of said species of mammal.

13. The plurality of biological materials according to claim 8 wherein said segment consists of nucleotides with sequence numbers ranging from 413 to 431 in said dhfr gene of said species of mammal.

14. The plurality of biological materials according to claim 1 wherein said dhfr gene included in said first vector is from a mammal which is in the same species as said CHO cell.

15. The plurality of biological materials according to claim 1 wherein said dhfr gene included in said first vector is from a mammal which is in a species different from the species of said CHO cell.

16. The plurality of biological materials according to claim 8 wherein said dhfr gene of said CHO cell is represented by SEQ ID NO: 2> and said dhfr gene of said species of mammal is represented by SEQ ID NO: 1.

17. The plurality of biological materials according to claim 8 wherein said DNA fragment consists of nucleotides characterizing SEQ ID NO: 3.

18. The plurality of biological materials according to claim 8 wherein said DNA fragment consists of nucleotides characterizing SEQ ID NO: 4.

19. The plurality of biological materials according to claim 8 wherein said DNA fragment consists of nucleotides characterizing SEQ ID NO: 5.

20. A plurality of biological materials for applying to at least a CHO cell for producing a species of protein, comprising:
a first vector including a dhfr gene of a species of mammal, and a gene of said species of protein; and
a second vector including a DNA fragment for transcripting at least a double-stranded RNA in said CHO cell after said second vector is applied to said CHO cell.
